(19) United States
(12) Plant Patent Application Publication
(10) Pub. No.: US 2014/0123354 P1
(43) Pub. Date: May 1, 2014

(54) Rhododendron rootstock named 'Rhodunter 150'

(50) ;

(71) Applicant: INKARHO GMBH, Bad Zwischenahn (DE)

(72) Inventor: Walter Preil, Ammersbek (DE)

(73) Assignee: Inkarho GmbH, Bad Zwischenahn (DE)

(21) Appl. No.: 13/694,107

(22) Filed: Oct. 30, 2012

(51) Int. Cl. A01H 5/00

(52) U.S. Cl. PLT/238

(57) ABSTRACT

A new and distinct Rhododendron hybrid plant used as a rootstock that exhibits a high lime tolerance and vigorous growth.

### BOTANICAL CLASSIFICATION

Rhododendron hybrid

### VARIETAL DENOMINATION

‘Rhodunter 150’

### BACKGROUND OF THE INVENTION

The present invention comprises a new and distinct cultivar of Rhododendron hybrid used as a rootstock known by the varietal name ‘Rhodunter 150’. The new variety was discovered in Ahrensburg, Germany. The new variety is the result of a planned breeding program between an unnamed and unpatented Rhododendron fortunei variety (female parent) and Rhododendron cv. ‘Cunningham's White’ (unpatented male parent). ‘Rhodunter 150’ exhibits leaves and flowers that are more similar to its female parent than to its male parent. Additionally, ‘Rhodunter 150 exhibits more vigorous growth when compared to its male parent. Further, the new variety differs from both parents in its improved tolerance to lime. In fact, such improved lime tolerance (no Fe-deficiency symptoms on soils up to 6.9 pH) distinguishes the new variety from other Rhododendron varieties known to the breeder and satisfied the purpose of the breeding program. ‘Rhodunter 150’ has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type:

FIG. 1 shows a view of the new variety grown to a flowering plant.

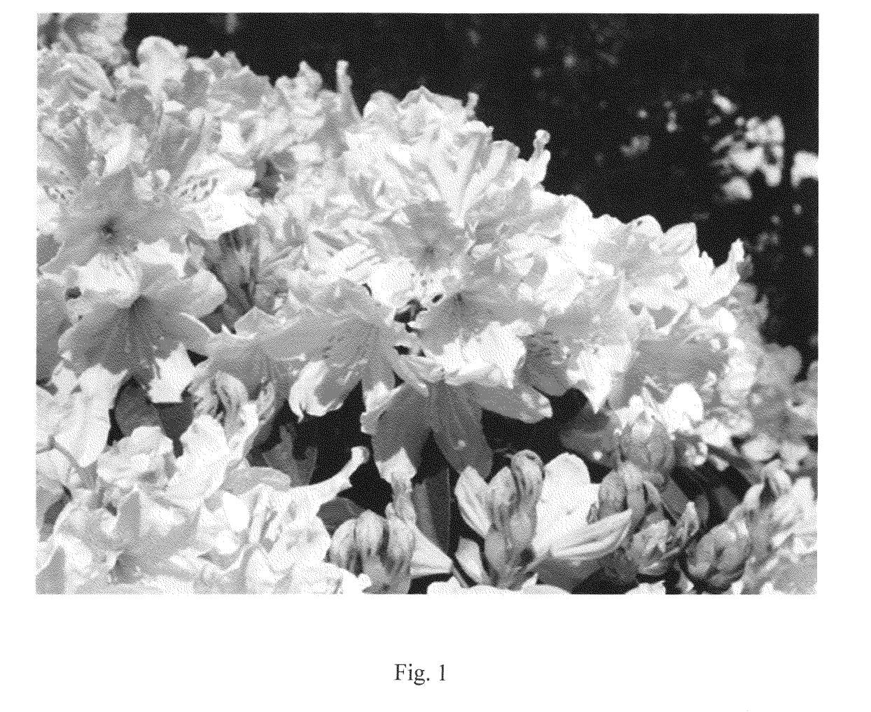

### DESCRIPTION OF THE PLANT

The following detailed description sets forth the characteristics of the new cultivar. The data which defines these characteristics were collected by asexual reproductions performed via cuttings or in vitro culture carried out in Ahrensburg, Germany. The color readings and measurements were taken outside under natural daylight on plants grown in containers. Color references are primarily to the R.H.S. Colour Chart of The Royal Horticultural Society of London.

### PLANT

Time to develop roots: About 90 days at 22-25° C.
   Time to produce a finished flowering plant from a rooted cutting.—About 4 years in the field.
   Rooting habit.—Spreading.
   Height.—50 cm. at 3 years.
   Diameter.—40 cm. at 3 years.
   Vigor.—Strong.
   Branching habit.—Compact.
   Disease, temperature, and drought resistance.—No observations made to date.
   Trunk.—Diameter: 3 cm. at 5 cm. above soil height at 4 years. Bark color: Green to reddish-brown. Texture: Hard. Pubescence: Absent. Form: Erect.
   Lateral branches.—Diameter: 5-7 mm. Internode length: 1-2 cm. Texture: Hard. Pubescence: Absent. Color: Green to reddish-brown.
   Leaves.—Arrangement: Alternate. Length: 15 cm. maximum. Width: 5.5 cm. maximum. Shape: Oblanceolate. Apex: Broad. Base: Tapering. Margin: Entire. Texture: Hard. Pubescence: Absent. Color: Upper surface: 147B for young leaves and 147A for mature leaves. Lower surface: 147C for both young and mature leaves. Margins: Entire. Venation pattern: Dense venation with a pronounced central vein. Vein color: Upper surface: 144A. Lower surface: 145C.
   Stipules.—None present.
   Bracts.—None present.
   Pedicels.—None present.
   Petiole.—Length: 3 cm. Diameter: 4-5 mm. Color: 147C.
Flower:
   Natural flowering season.—Beginning of May until the end of May in Northern Germany.
   Form.—Round spike.
   Lastingness of bloom.—From one to two weeks.
   Average number of blooms on a plant.—Approximately 20 on a plant with a diameter of 40 cm.
   Fragrance.—Typical of Rhododendron fortunei.
   Buds (described before bursting).—Shape: Oblong. Width: 20 mm. Length: 40 mm. Color: 34C.
   Spike.—Number of flowers per spike: 10-15. Length: 15 cm. Diameter: 15 cm.
   Corolla petals.—Form: Zygomorphic. Shape: Widely funnel-shaped. Number: 7. Length: Approximately 55 mm. Width: Approximately 50 mm. Margin: Undulate. Texture: Wax-like. Color (both surfaces): 11B when opening and fully opened.
   Corolla throat.—Tube length: Approximately 20 mm. Color: 1C. Texture: Wax-like.
   Sepals.—Fused.
Reproductive organs:
   Stamens.—Number: 14.
   Filament length.—Approximately 30 mm.
   Anthers.—Shape: Normal Rhododendron type. Color: Yellow. Length: 5 mm.
   Pollen.—Amount of pollen: Average. Color of pollen: Light brown.
   Pistil.—Number: 1. Length: Approximately 35 mm. Style: Color: Yellow. Length: Approximately 30 mm.; filiform. Stigma: Shape: Undivided. Color: Brown.
Seeds:
   Production.—Seed capsules mature in November when pollinated in May.
   Length.—2 mm.
   Color.—Brown.
   Number.—Approximately 200 per capsule.

I claim:

1. A new and distinct variety of Rhododendron hybrid plant, as is herein illustrated and described.
